(19) United States
(12) Patent Application Publication
(10) Pub. No.: US 2013/0338381 A1
(43) Pub. Date: Dec. 19, 2013

(54) Crude Reaction Product Comprising Dianhydro Sugar Alcohol and Method for Preparing the Same

(71) Applicant: CHEIL INDUSTRIES INC., Gumi-si (KR)

(72) Inventors: Man Suk Kim, Seongnam-si (KR); Sun Dae Kim, Seongnam-si (KR); Sung Hee Ahn, Seoul-si (KR); Sang Hyun Hong, Seongnam-si (KR)

(73) Assignee: CHEIL INDUSTRIES INC., Gumi-si (KR)

(21) Appl. No.: 13/798,274

(22) Filed: Mar. 13, 2013

(30) Foreign Application Priority Data
Jun. 13, 2012 (KR) 10-2012-0063242
Nov. 16, 2012 (KR) 10-2012-0130383

(51) Int. Cl. C07D 493/04

(52) U.S. Cl. CPC C07D 493/04
USPC 549/464

(57) ABSTRACT

A crude reaction product includes: (A) about 90 to 100% by weight of a dianhydro sugar alcohol in a solid form and (B) about 0 to about 10% by weight of a reaction byproduct in a solid form. The reaction product is prepared by the steps of (a) preparing a monoanhydro sugar alcohol by reacting a sugar alcohol in the presence of a first cyclization catalyst and (b) preparing a dianhydro sugar alcohol by reacting the monoanhydro sugar alcohol in the presence of a second catalyst.

### CROSS-REFERENCE TO RELATED APPLICATIONS

This application claims priority from Korea Patent Application Nos. 10-2012-0063242, filed on Jun. 13, 2012, and 10-2012-0130383, filed on Nov. 16, 2012, in the Korean Intellectual Property Office, the disclosure of each of which is incorporated herein by reference in its entirety.

### FIELD OF THE INVENTION

This invention relates to a crude reaction product comprising a dianhydro sugar alcohol.

### BACKGROUND OF THE INVENTION

The term of sugar alcohol refers to an alcohol with more than two hydroxyl groups formed by reducing the carbonyl group of a monosaccharide or compounds thereof. Examples of sugar alcohols include erythritol (C₄), threitol (C₄), arabitol (C₅), xylitol (C₅), mannitol (C₆), sorbitol (C₆), iditol (C₆), and the like which depend on the number of carbon therein.

Sugar alcohols are not only being used in various fields by themselves, but they are also being prepared into monoanhydro or dianhydro sugars. Monoanhydro and dianhydro sugars can be prepared as a compound with one or two rings by means of cyclodehydration of a sugar alcohol. For example, isosorbide has two rings prepared by cyclodehydration of sorbitol and can be used as a monomer of polyester, polycarbonate and the like.

U.S. Pat. No. 6,818,781 discloses a method for preparing of a dianhydro or monoanhydro sugar alcohol using sulfuric acid as a cyclization catalyst. However, this method can achieve only around 80% of yield and also can generate about 15 to 20% of oligomer or carbonized byproducts. In addition, a very complicated post processing may be required to obtain monomers which can be used in the polymerization of macromolecules.

U.S. Pat. No. 7,982,059 discloses a method for preparing of a dianhydro sugar alcohol by using zeolite as a cyclization catalyst. However, decarboxylation of a “C—O” unit may occur using this method, and therefore other sugar alcohols (xylitol) without a ring can be generated as a reaction byproduct.

### SUMMARY OF THE INVENTION

The present invention provides a crude reaction product comprising a dianhydro sugar alcohol. The crude reaction product can include a maximum amount of dianhydro sugar alcohol and/or a minimum amount of reaction byproducts (that is, the invention can help maximize the amount of dianhydro sugar alcohol and/or minimize the amount of reaction byproducts in the crude reaction product).

The present invention also provides a method for preparing the crude reaction product comprising a dianhydro sugar alcohol that can maximize the amount of dianhydro sugar alcohol and/or minimize the amount of reaction byproducts. The crude reaction product can be produced by conducting two cyclization reaction steps using a specific catalyst at each step.

A crude reaction product in accordance with this invention comprises (A) about 90 to about 100% by weight of a dianhydro sugar alcohol in a solid form and (B) about 0 to about 10% by weight of a reaction byproduct in a solid form.

The reaction byproduct (B) can include about 0 to about 5% by weight of an oligomeric compound, polymeric compound, or a combination thereof in a solid form. The reaction byproduct (B) can include about 0 to about 0.01% by weight of a sugar alcohol in a solid form. The reaction byproduct (B) can include less than about 0.01% by weight of a sorbitol, less than about 0.1% by weight of a 4-sorbitan, and less than about 2% by weight of a xylitol.

The dianhydro sugar alcohol (A) comprises two hetero rings.

A method for preparing of a crude reaction product comprising a dianhydro sugar alcohol comprises (a) the step of preparing a monoanhydro sugar alcohol by reacting sugar alcohol in the presence of a first cyclization catalyst and (b) the step of preparing a dianhydro sugar alcohol by reacting the monoanhydro sugar alcohol in the presence of a second cyclization catalyst.

The first cyclization catalyst includes two types of acid catalysts and the second cyclization catalyst is a heterogeneous catalyst. The two types of acid catalysts can be para-toluene sulfonic acid and phosphinic acid and the heterogeneous catalyst can be a zeolite.

The step of preparing the monoanhydro sugar alcohol (a) can be conducted at a temperature of about 100 to about 130° C., and the step of preparing the dianhydro sugar alcohol (b) can be conducted at a temperature about 100 to about 150° C. In addition, the step of preparing the monoanhydro sugar alcohol (a) can be conducted for about 10 to about 20 hours, and the step of preparing the dianhydro sugar alcohol can be conducted for about 10 to about 20 hours.

The step of preparing the monoanhydro sugar alcohol (a) can be conducted under a vacuum or nitrogen gas at normal atmospheric pressure conditions, and the step of preparing the dianhydro sugar alcohol (b) can be conducted under a vacuum or in nitrogen gas at normal atmospheric pressure conditions.

The sugar alcohol can be an aqueous solution.

The method can use about 0.1 to about 5 parts by weight of the first cyclization catalyst, and about 0.1 to about 5 parts by weight of the second cyclization catalyst based on about 100% by weigh of the sugar alcohol in a solid form.

The method for preparing of the crude reaction product comprising the dianhydro sugar alcohol of the present invention can further comprise a post processing step (c) of the crude reaction product. The post processing step (c) can comprise distillation and/or re-crystallization process.

### BRIEF DESCRIPTION OF THE FIGURE

FIG. 1 is a brief diagram of the reaction process for comparing the existing technology with the method for preparing of a dianhydro sugar alcohol of the present invention

### DETAILED DESCRIPTION OF THE INVENTION

The present invention now will be described more fully hereinafter in the following detailed description of the invention in which some but not all embodiments of the invention are described. Indeed, this invention may be embodied in many different forms and should not be construed as limited to the embodiments set forth herein; rather, these embodiments are provided so that this disclosure will satisfy applicable legal requirements.

Crude Reaction Product

A crude reaction product of the present invention comprises: (A) about 90 to about 100% by weight of a dianhydro sugar alcohol in a solid form and (B) about 0 to about 10% by weight of a reaction byproduct in a solid form.

The term crude reaction product means a mixture comprising the target product and the reaction byproducts obtained at the time of completion of the chemical reaction.

The term monoanhydro sugar alcohol refers to compound in which a ring is formed by removing a water molecule from a sugar alcohol.

The crude reaction product (A) of the present invention comprises about 90 to 100% by weight of the dianhydro sugar alcohol in a solid form. In some embodiments, the crude reaction product can include a dianhydro sugar alcohol (A) in a solid form in an amount of about 90, 91, 92, 93, 94, 95, 96, 97, 98, 99, or 100% by weight, based on 100% by weight of the crude reaction product. Further, according to some embodiments of the present invention, the amount of the dianhydro sugar alcohol (A) in a solid form can be in a range from about any of the foregoing amounts to about any other of the foregoing amounts.

Examples of dianhydro sugar alcohols (A) include without limitation isosorbide, isomannide, isoidide, and the like and combinations thereof. Isosorbide, isomannide, and isoidide are expressed by the following chemical formulas.

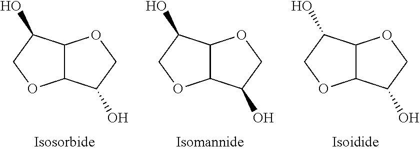

The dianhydro sugar alcohol (A) has two hetero rings. As used herein, the term hereto ring refers to a C5-C20 cycloalkyl structure, wherein at least one of the carbon atoms of the ring is replaced with a heteroatom such as oxygen. For example, the dianhydro sugar alcohol (A) may have two hetero rings, each comprising five atoms, wherein the hetero ring can comprise one oxygen atom and four carbon atoms (tetrahydrofuran). In addition, two of the hetero rings can share two of the carbon atoms.

The crude reaction product of the present invention comprises about 0 to about 10% by weight of the reaction byproducts (B) in a solid form. In some embodiments, the crude reaction product can include the reaction byproducts (B) in a solid form in an amount of 0 (no reaction byproducts (B) in a solid form are present), about 0 (reaction byproducts in a solid form are present), 1, 2, 3, 4, 5, 6, 7, 8, 9, or 10% by weight, based on 100% by weight of the crude reaction product. Further, according to some embodiments of the present invention, the amount of the reaction byproducts (B) in a solid form can be in a range from about any of the foregoing amounts to about any other of the foregoing amounts.

Examples of the reaction byproducts (B) include without limitation oligomer form compounds, polymer form compounds and the like, and combinations thereof. The present invention can substantially reduce costs during post processing steps, such as purification, by minimizing the amount of reaction byproduct (B).

In exemplary embodiments, the reaction byproduct (B) comprises about 0 to about 5% by weight, for example about 0 to about 1% by weight of an oligomer compound, a polymer compound, or a combination thereof in a solid form. In some embodiments, the crude reaction product can include an oligomer compound, a polymer compound, or a combination thereof in a solid form in an amount of 0 (no oligomer compound, polymer compound, or combination thereof in a solid form are present), about 0 (an oligomer compound, a polymer compound, or a combination thereof in a solid form is present), 1, 2, 3, 4, or 5% by weight, based on 100% by weight of the crude reaction product. Further, according to some embodiments of the present invention, the amount of an oligomer compound, a polymer compound, or a combination thereof in a solid form can be in a range from about any of the foregoing amounts to about any other of the foregoing amounts.

In exemplary embodiments, the reaction byproduct (B) comprises about 0 to about 0.01% by weight, for example about 0 to about 0.001% by weight, of the sugar alcohol in a solid form. This means that the crude reaction product as a practical matter includes a minimal amount, or does not include, the sugar alcohol. The sugar alcohol can be the same or a different from one of the first starting substances. For example, if the starting substance is sorbitol, the final sugar alcohol can be sorbitol, xylitol, or a mixture thereof.

In exemplary embodiments, the reaction byproduct (B) comprises less than about 0.1% by weight of sorbitol, less than about 0.1% by weight of 1,4-sorbitan, and less than about 2% by weight of xylitol.

The crude reaction byproduct of the present invention can further comprise about 0 to about 10% by weight of an intermediate product in a solid form. In some embodiments, the crude reaction product can include an intermediate product in a solid form in an amount of 0 (no intermediate products in a solid form are present), about 0 (intermediate products in a solid form are present), 1, 2, 3, 4, 5, 6, 7, 8, 9, or 10% by weight, based on 100% by weight of the crude reaction product. Further, according to some embodiments of the present invention, the amount of the intermediate product in a solid form can be in a range from about any of the foregoing amounts to about any other of the foregoing amounts.

As used herein, the term “intermediate product” means a monoanhydro sugar alcohol. Examples of the monanhydro sugar alcohols include without limitation 1,4-sorbitan, 3,6-sorbitan, 2,5-mannitan, 2,5-iditan, and the like, and combinations thereof.

The crude reaction product of the present invention can further comprise about 0 to about 5% by weight of two types of acid catalysts, heterogeneous catalyst, or combinations thereof. In some embodiments, the crude reaction product can include two types of acid catalysts, heterogeneous catalyst, or combinations thereof in an amount of 0 (no two types of acid catalysts, heterogeneous catalyst, or combinations thereof are present), about 0 (two types of acid catalysts, heterogeneous catalyst, or combinations thereof are present), 1, 2, 3, 4, or 5% by weight, based on 100% by weight of the crude reaction product. Further, according to some embodiments of the present invention, the amount of the two types of acid catalysts, heterogeneous catalyst, or combinations thereof can be in a range from about any of the foregoing amounts to about any other of the foregoing amounts.

Examples of the two types of catalysts include without limitation p-toluene sulfonic acid, phosphinic acid, and the like, and combinations thereof. An example of a heterogeneous catalyst includes without limitation a zeolite.

Method for Preparing of Crude Reaction Product Comprising a Dianhydro Sugar Alcohol

A method for preparing of the crude reaction product comprising a dianhydro sugar alcohol of the present invention comprises (a) a step of preparing a monoanhydro sugar alcohol by reacting sugar alcohol in the presence of a first cyclization catalyst (the first cyclization reaction) and (b) a step of preparing a dianhydro sugar alcohol by reacting the monoanhydro sugar alcohol in the presence of a second cyclization catalyst (the second cyclization reaction).

The present invention can maximize the yield of the dianhydro sugar alcohol as compared with existing one step cyclization methods using sulfuric acid and zeolite and/or further can minimize the amount of the reaction byproducts.

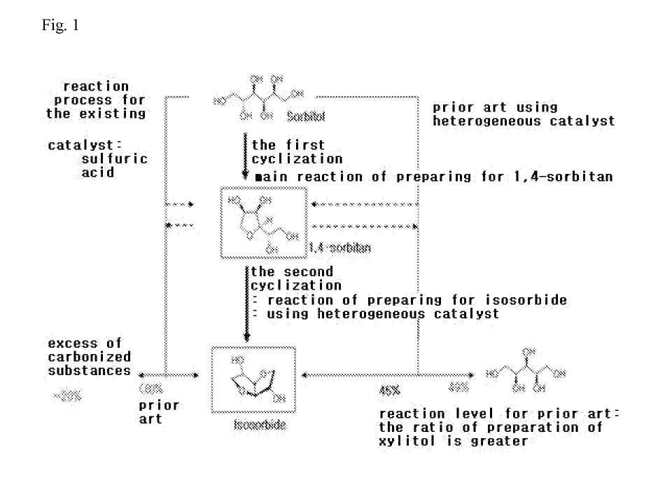

FIG. 1 is a brief diagram of the reaction process for comparing the existing technology with the method for preparing of a dianhydro sugar alcohol of the present invention. On the left side of FIG. 1, if sulfuric acid is used as a cyclization catalyst in a single step, yield of the dianhydro sugar alcohol can be low and carbonized substances can be produced in excess. While, on the right side, if a heterogeneous catalyst (zeolite) is used as the cyclization catalyst in the single step reaction, the reaction can have an excessive amount of xylitol as the byproduct.

The reaction product of step (a) of the method for preparing the monoanhydro sugar alcohol can further comprise a dianhydro sugar alcohol in addition to the monoanhydro sugar alcohol. For example, about 20 to about 50% by weight of a monoanhydro sugar alcohol in a solid form and about 50 to about 80% by weight of a dianhydro sugar alcohol in a solid form can be prepared by means of the first cyclization reaction. In addition, about 0 to about 10% by weight of a byproduct in a solid form can be further prepared by means of the first cyclization reaction.

In some embodiments, the reaction product of step (a) can include monoanhydro sugar alcohol in a solid form in an amount of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 wt % by weight. Further, according to some embodiments of the present invention, the amount of monoanhydro sugar alcohol in a solid form can be in a range from about any of the foregoing amounts to about any other of the foregoing amounts.

In some embodiments, the reaction product of step (a) can include dianhydro sugar alcohol in a solid form in an amount of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 wt % by weight. Further, according to some embodiments of the present invention, the amount of dianhydro sugar alcohol in a solid form can be in a range from about any of the foregoing amounts to about any other of the foregoing amounts.

In some embodiments, the reaction product of step (a) can include a byproduct in a solid form in an amount of 0 (the byproduct is not present), about 0 (the byproduct is present), 1, 2, 3, 4, 5, 6, 7, 8, 9, or 10 wt % by weight. Further, according to some embodiments of the present invention, the amount of byproduct in a solid form can be in a range from about any of the foregoing amounts to about any other of the foregoing amounts.

The starting substances in the step of preparing the dianhydro sugar alcohol (b) can further comprise a dianhydro sugar alcohol in addition to the monoanhydro sugar alcohol. For example, the starting substance in the step of preparing the dianhydro sugar alcohol (b) can comprise about 20 to about 50% by weight of the monoanhydro sugar alcohol in a solid form and about 50 to about 80% by weight of the dianhydro sugar alcohol in a solid form.

In some embodiments, the starting substance in the step of preparing the dianhydro sugar alcohol (b) can include monoanhydro sugar alcohol in a solid form in an amount of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 wt % by weight. Further, according to some embodiments of the present invention, the amount of monoanhydro sugar alcohol in a solid form can be in a range from about any of the foregoing amounts to about any other of the foregoing amounts.

In some embodiments, the starting substance in the step of preparing the dianhydro sugar alcohol (b) can include dianhydro sugar alcohol in a solid form in an amount of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 wt % by weight. Further, according to some embodiments of the present invention, the amount of dianhydro sugar alcohol in a solid form can be in a range from about any of the foregoing amounts to about any other of the foregoing amounts.

The step of preparing the monoanhydro sugar alcohol (a) can be conducted at a temperature of about 100 to about 130° C. In this case, the composition ratio and color of the monoanhydro sugar alcohol can be excellent while minimizing the byproducts.

The step of preparing the monoanhydro sugar alcohol (a) can be conducted for about 10 to about 20 hours. In this case, the color of the monoanhydro sugar alcohol can be excellent.

The step of preparing the monoanhydro sugar alcohol (a) can be conducted until the sugar alcohol content in a solid form becomes about 0 to about 1% by weight, for example until the sugar alcohol content in a solid form becomes 0% by weight. In this case, an amount of the byproducts derived from the sugar alcohol can be minimized.

The step of preparing the dianhydro sugar alcohol (b) can be conducted at a temperature of about 100 to about 150° C. In this case, the composition and color of the dinoanhydro sugar alcohol can be excellent.

The step of preparing the dianhydro sugar alcohol (b) can be conducted for about 10 to about 20 hours. In this case, the color of dianhydro sugar alcohol and activity of the catalyst can be excellent.

The step of preparing the dianhydro sugar alcohol (b) can be conducted until the sugar alcohol content in a solid form becomes about 0 to about 1% by weight, for example until the sugar alcohol content in a solid form becomes 0% by weight.

The step of preparing the monoanhydro sugar alcohol (a) can be conducted under vacuum or in nitrogen gas at normal atmospheric pressure condition. In this case, the process time can be reduced. In addition, the step of preparing the dianhydro sugar alcohol (b) can be conducted under vacuum or in nitrogen gas at normal atmospheric pressure condition. In this case, while reducing the process time, the color of the dianhydro sugar alcohol can also be excellent.

The first cyclization catalysts of the present invention are two types of acid catalysts, and the second cyclization catalyst is a heterogeneous catalyst. In exemplary embodiments, the two types of acid catalysts are p-toluene sulfonic acid and phosphinic acid. Examples of the heterogeneous catalysts include without limitation formed metals, multi-porous inorganic substances, and the like. In exemplary embodiments, a zeolite can be used. When these catalysts are used, the second cyclization reaction can be carried out in continuation.

An aqueous sugar alcohol can be used to reduce the process cost. For example, about 50 to about 90% by weight of a sugar alcohol in aqueous form can be used. In some embodiments, an aqueous sugar alcohol can be used in an amount of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 wt % by weight. Further, according to some embodiments of the present invention, the amount of aqueous sugar alcohol can be in a range from about any of the foregoing amounts to about any other of the foregoing amounts.

About 0.1 to about 5 parts by weight of the first cyclization catalyst can be used as in a solid form based on about 100 parts by weight of the sugar alcohol in a solid form. In some embodiments, the first cyclization catalyst can be used in an amount of about 0.1, 0.2, 0.3, 0.4, 05, 0.6, 0.7, 0.8, 0.9, 1, 2, 3, 4, or 5 parts by weight. Further, according to some embodiments of the present invention, the amount of the first cyclization catalyst can be in a range from about any of the foregoing amounts to about any other of the foregoing amounts.

About 0.1 to about 5 parts by weight of the second cyclization catalyst can be used as in a solid form based on about 100 parts by weight of the monoanhydro sugar alcohol in a solid form. In some embodiments, the second cyclization catalyst can be used in an amount of about 0.1, 0.2, 0.3, 0.4, 05, 0.6, 0.7, 0.8, 0.9, 1, 2, 3, 4, or 5 parts by weight. Further, according to some embodiments of the present invention, the amount of the second cyclization catalyst can be in a range from about any of the foregoing amounts to about any other of the foregoing amounts.

The method for preparing of the crude reaction product comprising dianhydro sugar alcohol can further comprise a post processing step (c) wherein the prepared crude reaction product is further processed. Examples of the post processing steps (c) comprise without limitation distillation, decoloration, extraction, purification, and the like. These processes can be easily carried out by a person having a common knowledge in the field to which the present invention relates. The post processing step can be carried out by selecting one or more process(es) from the aforesaid processes.

The present invention is described in more detail by the following examples. However, the following examples are to be used to merely exemplify this invention and are not intended to limit the scope of protection of the present invention.

### EXAMPLES

### Example 1

### The First Cyclization Reaction

In a about 20 mL vial containing about 5 g of about 70% sorbitol aqueous solution, the catalysts p-toluene sulfonic acid (p-TsOH) (about 9.1 mg, about 0.26% by weight) and H₃PO₂ (about 35 mg, 1.0% by weight) are added. While nitrogen (N₂) gas flows into the system and is discharged through the outlet, this mixture is stirred for about 16 hours by gradually increasing the temperature to about 115° C. NMR measurement for the formed crude compound using AcPh (acetophenone) as an internal standard revealed that the formed crude compound is synthesized into 1,4-sorbitan (about 35%) and isosorbide (about 61%) as the main products.

The Second Cyclization Reaction

After adding the catalyst zeolite (about 630 mg, about 18% by weight of sorbitol) into the product of the first cyclization reaction, the mixture is stirred for three hours under the condition of compressed nitrogen (about 60.0 bar) and by raising the temperature (about 250° C.). The crude compound so produced is measured by NMR using AcPh (acetophenone) as an internal standard and it is found that isosorbide (about 98%) is formed as the main ingredient.

### Comparative Example 1

Comparative Example 1 is conducted in the same manner as described in Example 1 except for sulfuric acid (about 35 mg, about 1% by weight) is added as a catalyst in the first cyclization reaction and the second cyclization reaction is not carried out.

### Comparative Example 2

Comparative Example is conducted in the same manner as described in Example 2 except for that the zeolite (about 630 mg, about 18% by weight of sorbitol) is added as a catalyst in the first cyclization reaction and the additional reaction is not carried out under the condition of compressed nitrogen (about 60.0 bar) and by raising the temperature (about 250° C.).

TABLE 1

| | | Examples | | | |
|---|---|---|---|---|---|
| | | First | Second | | |
| | Before | cyclization | cyclization | Comparative | Comparative |
| | reaction | reaction | reaction | Example 1 | Example 2 |
| sorbitol | 100 | 0 | 0 | 0 | 15.5 |
| 1,4-sorbital | 0 | 35 | 0 | 0 | 0 |
| Isosorbide | 0 | 61 | 98 | 78 | 21 |
| Xylitol | 0 | 0 | 1 | 0 | 54.7 |
| Unknown | 0 | 4 | 1 | 22 | 8 |

[Unit: % by weight (in a solid form)]

From Table 1, it is clear that the amount of isosorbide can be maximized through the two steps of cyclization reactions using a specific cyclization catalyst for each step as in the Examples, while the byproduct which is expressed as Unknown can be minimized

Comparative example 1 shows that an excessive amount of the unknown byproduct is formed in a single step of cyclization in which sulfuric acid is used. In addition, in Comparative Example 2, a slightly excessive amount of the unknown byproduct is formed by a single step of cyclization in which a zeolite is used. An excessive amount of xylitol is also formed and a slightly excessive amount of sorbitol is not reacted in Comparative Example 2.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation, the scope of the invention being defined in the claims.

That which is claimed is:

1. A crude reaction product, comprising:
(A) about 90 to about 100% by weight of a dianhydro sugar alcohol in a solid form; and
(B) about 0 to about 10% by weight of a reaction byproduct in a solid form.

2. The crude reaction product of claim 1, wherein the reaction byproduct (B) comprises about 0 to about 5% by weight of an oligomer compound, a polymer compound, or a combination thereof in a solid form.

3. The crude reaction product of claim 1, wherein the reaction byproduct (B) comprises about 0 to about 0.01% by weight of a sugar alcohol in a solid form.

4. The crude reaction product of claim 1, wherein the reaction byproduct (B) comprises less than about 0.1% by weight of sorbitol, less than about 0.1% by weight of 1,4-sorbitan, and less than about 2% by weight of xylitol.

5. The crude reaction product of claim 1, wherein the dianhydro sugar alcohol (A) has two hetero rings.

6. A method for preparing of the crude reaction product comprising a dianhydro sugar alcohol comprising the steps of:
(a) preparing a monoanhydro sugar alcohol by reacting a sugar alcohol in the presence of a first cyclization catalyst; and
(b) preparing a dianhydro sugar alcohol by reacting the monoanhydro sugar alcohol in the presence of a second cyclization catalyst.

7. The method of claim 6, wherein the first cyclization catalysts includes two types of acid catalysts and the second cyclization catalyst is a heterogeneous catalyst.

8. The method of claim 7, wherein the two types of acid catalysts are p-tolulene sulfonic acid and phosphinic acid, and said heterogeneous catalyst is a zeolite.

9. The method of claim 6, wherein the step of preparing the monoanhydro sugar alcohol (a) is conducted at a temperature of at about 100 to about 130° C. and the step of preparing the dianhydro sugar alcohol (b) is conducted at a temperature of about 100 to about 150° C.

10. The method of claim 6, wherein the step of preparing the monoanhydro sugar alcohol (a) is conducted for about 10 to about 20 hours and the step of preparing the dianhydro sugar alcohol (b) is conducted for about 10 to about 20 hours.

11. The method of claim 10, wherein the step of preparing the monoanhydro sugar alcohol (a) is conducted under vacuum or in nitrogen gas at normal atmospheric pressure condition and the step of preparing the dianhydro sugar alcohol (b) is conducted under vacuum or nitrogen gas at normal atmospheric pressure condition.

12. The method of claim 7, wherein the sugar alcohol is an aqueous solution, about 0.1 to about 5 parts by weight of the first cyclization catalyst is used in a solid form based on about 100 parts by weight of the sugar alcohol in a solid form, and about 0.1 to about 5 parts by weight of the second cyclization catalyst is used in a solid form based on about 100 parts by weight of the monoanhydro sugar alcohol in a solid form.

13. The method of claim 6, further comprising a post processing step (c) of the crude reaction product.

14. The method of claim 13, wherein the post processing step (c) comprises a distillation or re-crystallization process.
